SEQUENCE LISTING (15 sequences)

SEQ ID NO: 1 - protein, 12 aa, synthetic construct
TKRKHPHRRKYR

SEQ ID NO: 2 - protein, 18 aa, synthetic construct
GTGSGAGTGSGAGTGSGA

SEQ ID NO: 3 - protein, 42 aa, synthetic construct
TKRKHPHRRKYRGTGSGAGTGSGAGTGSGATKRKHPHRRKYR

SEQ ID NO: 4 - protein, 55 aa, synthetic construct
SMHPGHLKGREIGMWYAKKQGQKNKEAERQERAVVHMDERREEQIVQLLNSVQAK

SEQ ID NO: 5 - protein, 12 aa, synthetic construct
AHPLTKPPKYHH

SEQ ID NO: 6 - RNA, 20 nt, synthetic construct
  source 1..20: mol_type = other RNA
gggttgcggagggtgggcct

SEQ ID NO: 7 - RNA, 20 nt, synthetic construct
  source 1..20: mol_type = other RNA
gaattgcggagaatgaacct

SEQ ID NO: 8 - DNA, 26 bp, synthetic construct
  source 1..26: mol_type = other DNA
ggggagggtggggagggtggggaagg

SEQ ID NO: 9 - DNA, 22 bp, synthetic construct
  source 1..22: mol_type = other DNA
agggagggcgctgggaggaggg

SEQ ID NO: 10 - DNA, 27 bp, synthetic construct
  source 1..27: mol_type = other DNA
tcgggttgcgggcgcagggcacgggcg

SEQ ID NO: 11 - DNA, 24 bp, synthetic construct
  source 1..24: mol_type = other DNA
ttagggttagggttagggttaggg

SEQ ID NO: 12 - DNA, 66 bp, synthetic construct
  source 1..66: mol_type = other DNA
gggttgcggagggtgggcctatagggttgcggagggtgggcctatagggttgcggagggtgggcct

SEQ ID NO: 13 - DNA, 66 bp, synthetic construct
  source 1..66: mol_type = other DNA
gaattgcggagaatgaacctatagaattgcggagaatgaacctatagaattgcggagaatgaacct

SEQ ID NO: 14 - DNA, 104 bp, synthetic construct
  source 1..104: mol_type = other DNA
  variation 46..81: note = m is a or c; n is a or g or c or t/u
cccgcctcgcgcccgccgtccctagctacctcctcctccaccgcamnnmnnmnnmnnmnnmnnmnnmnnmnnmnnmnnmnncataaatatcacctccttaatcc

SEQ ID NO: 15 - DNA, 21 bp, synthetic construct
  source 1..21: mol_type = other DNA
cccgcctcccgccccccgtcc

(19) United States
(12) Patent Application Publication
(10) Pub. No.: US 2024/0255515 A1
(43) Pub. Date: Aug. 1, 2024

(54) G4-SPECIFIC PEPTIDES AND METHODS OF IDENTIFYING THE SAME

(71) Applicant: CITY UNIVERSITY OF HONG KONG, Kowloon (HK)

(72) Inventors: Chun Kit KWOK, Kowloon (HK); Xi MOU, Kowloon (HK)

(21) Appl. No.: 18/427,626

(22) Filed: Jan. 30, 2024

Related U.S. Application Data
(60) Provisional application No. 63/482,258, filed on Jan. 30, 2023.

(51) Int. Cl. G01N 33/68; C07K 7/64; C07K 14/00; C12Q 1/6869; A61K 38/00

(52) U.S. Cl. CPC G01N 33/6803; C07K 7/64; C07K 14/001; C12Q 1/6869; A61K 38/00

(57) ABSTRACT

Disclosed herein is a method of identifying a peptide specific to a target nucleic acid from a DNA library. According to some embodiments of the present disclosure, the method comprises, in vitro transcribing the DNAs of the DNA library into RNAs, producing RNA-cDNA-peptide conjugates from the RNAs by using a puromycin linker, selecting cDNAs from the RNA-cDNA-peptide conjugates exhibiting binding affinity to the target nucleic acid, and then identifying the peptide based on the selected cDNA. Also disclosed herein are the peptide identified by the present method, and uses thereof in treating telomerase-related diseases.

### CROSS-REFERENCE TO RELATED APPLICATIONS

This application relates to and claims the benefit of U.S. Provisional Application No. 63/482,258 filed Jan. 30, 2023; the content of the application is incorporated herein by reference in its entirety.

### STATEMENT REGARDING PRIOR DISCLOSURES BY THE INVENTOR OR A JOINT INVENTOR UNDER 37 C.F.R. 1.77(B)(6)

Most of the subject matter of the invention described in the present application was published by the inventors, Chun Kit Kwok and Xi Mou in an article titled “Peptides Selected by G4-mRNA Display-Seq Enable RNA G-Quadruplex Recognition and Gene Regulation.” The article was published online by Journal of the American Chemical Society on Aug. 15, 2023. Therefore, the publication was made by and/or originated from all member of the inventive entity of the present invention less than one year before the filing date of the present application. A copy of the article is provided in a concurrently filed Information Disclosure Statement pursuant to the guidance of 78 Fed. Reg. 11076 (Feb. 14, 2013).

### SEQUENCE LISTING XML

The present application is being filed along with a Sequence Listing XML in electronic format. The Sequence Listing XML is provided as an XML file entitled HP0290US_SEQ_AF, created Nov. 8, 2023, which is 15 Kb in size. The information in the electronic format of the Sequence Listing XML is incorporated herein by reference in its entirety.

### BACKGROUND OF THE INVENTION

### 1. Field of the Invention

The present disclosure in general relates to the field of G-quadruplex (G4)-specific peptides. More particularly, the present disclosure relates to a method for identifying peptides, which recognize and bind to target nucleic acids having G4 structures.

### 2. Description of Related Art

Guanine (G)-rich sequences can self-assemble into non-canonical structure motifs referred to as G4s. These special structures are stabilized by metal cations such as potassium ion (K⁺) and sodium ion (Na⁺) rather than lithium ion (Li⁺). Over the past few decades, G4s have been reported to regulate diverse cellular processes, including gene expression, ribonucleic acid (RNA) metabolism, epigenetic states, and associated diseases such as neurological diseases and cancers. The structural conformations and biological significance of G4s make them promising therapeutic targets for drug development. However, the study of G4s has been limited by the lack of efficient and specific targeting tools for their structural recognition and gene manipulation.

Current major approaches used for G4 targeting include G4-specific chemicals and G4-specific antibodies. While these molecules exhibit great specificity and affinity for G4s, only limited cases have been reported to have the potential to differentiate between RNA G4s (rG4s) and deoxyribonucleic acid (DNA) G4s (dG4s). To address this, it is important to develop innovative platforms and explore new G4 targeting tools such as G4-specific peptides. To date, most of reports regarding the G4-binding peptides, such as RHAU23, RHAU53, and RGG motif, were derived from endogenous proteins. While a few de novo peptides and one nanobody targeting dG4s have been developed recently using phage display, their affinity and selectivity to recognize rG4s were not examined, and their abilities to regulate rG4-mediated gene activity remain unexplored.

In view of the foregoing, there is a continuing interest in developing a novel method for developing G4-targeting peptides.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the disclosure is directed to a method of identifying a peptide specific to a target nucleic acid from a DNA library, wherein the DNA library comprises a first plurality of DNAs respectively having randomized sequences. According to the embodiments of the present disclosure, the method comprises,
(a) producing a plurality of RNAs respectively corresponding to the first plurality of DNAs via in vitro transcription;
(b) attaching a puromycin molecule to the 3′ end of each of the plurality of RNAs of step (a) via a linker;
(c) subjecting the product of step (b) to in vitro translation to produce a plurality of RNA-peptide conjugates;
(d) subjecting the product of step (c) to reverse transcription to produce a first plurality of RNA-complementary DNA (cDNA)-peptide conjugates;
(e) mixing the product of step (d) with the target nucleic acid to identify a second plurality of RNA-cDNA-peptide conjugates bound to the target nucleic acid;
(f) eluting the cDNAs from the second plurality of RNA-cDNA-peptide conjugates of step (e);
(g) subjecting the product of step (f) to polymerase chain reaction (PCR) to produce a second plurality of DNAs;
(h) repeating steps (a) to (g) at least 5 times, in which except in the first run of steps (a) to (g), the in vitro transcription in each repeat is performed by using the product of step (g) in a previous run of steps (a) to (g), so as to produce a selected cDNA eluted from the second plurality of RNA-cDNA-peptide conjugates of step (f) in the final repeat; and
(i) determining the nucleotide sequence of the selected cDNA of step (h) so as to identify the peptide specific to the target nucleic acid.

In general, the target nucleic acid may be DNA or RNA. According to some embodiments of the present disclosure, the target nucleic acid has a G4 structure.

Optionally, the target nucleic acid of step (e) is linked to a biotin molecule. According to some exemplary embodiments, the present method further comprises (e-1) capturing the product of step (e) via a streptavidin-coated bead prior to step (f). In some optional embodiments, the method further comprises (e-2) subjecting the product of step (e-1) to centrifugation or magnetic field prior to step (f).

Optionally, the present method further comprises (d-1) mixing the product of step (d) with the streptavidin-coated bead prior to step (e). According to certain optional embodiments, the method further comprises (d-2) subjecting the product of step (d-1) to centrifugation or magnetic field prior to step (e).

Also disclosed herein a peptide identified by the present method, and the modified forms thereof. According to the embodiments of the present disclosure, the peptide is designated as “pep11”, and comprises the amino acid sequence of “TKRKHPHRRKYR” (SEQ ID NO: 1).

According to some embodiments, the modified pep11 is present in cyclic form. In the embodiments, the pep11 in cyclic form is designated as “cyclic pep11”.

According to certain embodiments, the modified pep11 is present in tandem form. In the embodiments, the pep11 in tandem form is designated as “tandem pep11” that in its structure comprises two binding domains linked via a peptide linker, wherein each of the binding domains comprises the amino acid sequence of SEQ ID NO: 1. According to one exemplary embodiment, the peptide linker comprises the amino acid sequence of “(GTGSGA)₃” (SEQ ID NO: 2); in this case, the tandem pep11 comprises the amino acid sequence of

(SEQ ID NO: 3)
“TKRKHPHRRKYRGTGSGAGTGSGAGTGSGATKRKHPHRRKYR”.

Another aspect of the present disclosure pertains to a method of treating a telomerase-related disease in a subject. The method comprises administering to the subject an effective amount of the peptide (i.e., pep11, cyclic pep11, or tandem pep11).

Examples of telomerase-related disease treatable with the present method include, but are not limited to, cancer, Hodgkin's disease, dyskeratosis congenital, and aging.

The subject is a mammal; preferably, a human.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings briefly discussed below.

FIG. 1 is a schematic diagram depicting the design of dual luciferase reporter plasmid according to Example 2.4 of the present disclosure.

FIGS. 2A to 2D are histograms respectively depicting the effects of tandem pep11 (FIG. 2A), cyclic pep11 (FIG. 2B), RHAU53 (FIG. 2C) and pep71 (FIG. 2D) on regulating rG4-medicated gene activity in cells according to Example 2.4 of the present disclosure. The error bar represents the standard deviation of four independent replicates; *, p<0.05; **, p<0.01; ***, p<0.001; NS: not significant.

In accordance with common practice, the various described features/elements are not drawn to scale but instead are drawn to best illustrate specific features/elements relevant to the present invention.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “G-quadruplex structure” (G4 structure) as used herein refers to a four-stranded helical nucleic acid structure comprising multiple stacked G-tetrads, each of which consists of four guanine bases that associate in a cyclical manner through Hoogsteen hydrogen bonds and are further stabilized, through coordination to a cation in the center. The body of stacked G-tetrads, comprising a total of 2-8 layers, is collectively referred to as the G-tetrad core. Each of the four guanine columns constituting the G-tetrad core can arise from a single (continuous column), two, or four (discontinuous column) separate guanine stretch/stretches. The term “parallel G-quadruplex”, as used herein, relates to a G-quadruplex structure wherein all four strands point in the same direction.

The term “transcription” is known in the art, and refers to a reaction during which a nucleic acid molecule (e.g., a DNA molecule) serving as a template and having a particular nucleotide sequence is read by an RNA polymerase so that the RNA polymerase produces a single-stranded RNA molecule. During transcription, the genetic information in a nucleic acid template is transcribed, and subsequently the transcribed RNA may be further translated into protein. As used herein, the term “in vitro transcription” refers to a reaction wherein RNA, in particular mRNA, is in vitro synthesized from DNA template in a cell-free system, e.g., appropriate cell extracts. According to some embodiments of the present disclosure, in vitro transcription is carried out by incubating reactants, including DNA template containing a promoter, RNA polymerase, nucleotide triphosphates (NTPs), reaction buffer (e.g., Tris or Tris-HCl), salt (e.g., MgCl₂), reducing agent (optionally; e.g., dithiothreitol, DTT) and RNase inhibitor (optionally), at an appropriate temperature (e.g., 37° C.) for a period of time.

As used herein, the term “reverse transcription” refers to a reaction in which an RNA template is reverse transcribed using a reverse transcriptase into a complementary DNA (cDNA) chain. A reverse transcription reaction usually includes, RNA template, reverse transcriptase, reaction buffer (e.g., Tris or Tris-HCl), salt, primers (e.g., oligo(dT) primers or random primers), deoxynucleoside triphosphates (dNTPs), reducing agent (optionally; e.g., dithiothreitol, DTT) and RNase inhibitor (optionally). Depending upon the context, the mixture can be either a complete or incomplete reverse transcription reaction mixture.

The term “in vitro translation” (also known as “cell-free protein synthesis”) as used herein refers to protein production in vitro using a cell-free translation system reconstituted from the purified components necessary for E. coli translation. For the purpose of carrying out an in vitro translation, various reactants, including RNA template, ribosome, transfer RNA (tRNA), aminoacyl-tRNA synthetase, initiation/elongation/termination factors, guanosine triphosphate (GTP), adenosine triphosphate (ATP) and ions (e.g., Mg²⁺ and/or K⁺), are required for a translation system. These reactants may be provided by lysates, which can be from prokaryotic or eukaryotic sources, depending on practical uses.

The term “treat” or “treatment” as used herein are intended to mean obtaining a desired pharmacological and/or physiologic effect. The effect may be prophylactic in terms of completely or partially preventing a disease or condition (e.g., a telomerase-related disease or condition, such as cancer, Hodgkin's disease, dyskeratosis congenital, or aging) or symptom thereof and/or therapeutic in terms of a partial or complete cure for a disease/condition and/or adverse effect attributable to the disease/condition. “Treatment” as used herein includes preventative (e.g., prophylactic), curative or palliative treatment of a disease/condition in a mammal, particularly human; and includes, (1) preventative (e.g., prophylactic), curative or palliative treatment of a disease or condition from occurring in an individual who may be pre-disposed to the disease but has not yet been diagnosed as having it; (2) inhibiting a disease or condition (e.g., by arresting its development); or (3) relieving a disease or condition (e.g., reducing symptoms associated with the disease or condition).

The term “effective amount” as referred to herein designate the quantity of a component which is sufficient to yield a desired response. For therapeutic purposes, the effective amount is also one in which any toxic or detrimental effects of the component are outweighed by the therapeutically beneficial effects. An effective amount of an agent is not required to cure a disease or condition but will provide a treatment for a disease or condition such that the onset of the disease or condition is delayed, hindered or prevented, or the disease or condition symptoms are ameliorated. The effective amount may be divided into one, two, or more doses in a suitable form to be administered at one, two or more times throughout a designated time period. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the structure of the compounds or its derivatives.

The term “subject” refers to a mammal including the human species that is treatable with methods of the present invention. The term “subject” is intended to refer to both the male and female gender unless one gender is specifically indicated.

### II. Description of the Invention

The present invention aims at providing a platform for identifying G4-targeting peptides. According to the embodiments of the present disclosure, the peptide identified by the present method exhibits binding affinity and/or specificity towards a target nucleic acid having a G4 structure (for example, the human telomerase RNA component (hTERC)), and is useful in regulating the expression and/or activity of the target nucleic acid. The peptide thus provides a potential means to treat a disease or condition (e.g., a telomerase-related disease or condition) associated with and/or caused by the target nucleic acid via regulating the its expression and/or activity.

Accordingly, the first aspect of the present disclosure is directed to a method of identifying a peptide specific to a target nucleic acid (such as a target DNA or RNA having a G4 structure, i.e., dG4 or rG4) from a DNA library. According to some embodiments of the present disclosure, the DNA library comprises a plurality of DNAs, each of which has a randomized nucleotide sequence (12 repeats of the nucleotide sequence “MNN”, wherein M is nucleotide “A” or “C”, and N is any of nucleotide “A”, “T”, “C” or “G”) in its sequence. The present method comprises the steps of,
(a) producing a plurality of RNAs respectively corresponding to the plurality of DNAs via in vitro transcription;
(b) attaching a puromycin molecule to the 3′ end of each RNAs of step (a) via a linker;
(c) subjecting the product of step (b) to in vitro translation to produce a plurality of RNA-peptide conjugates (hereinafter as “RNA/peptide conjugates”);
(d) subjecting the product of step (c) to reverse transcription to produce a plurality of RNA-cDNA-peptide conjugates (hereinafter as “RNA/cDNA/peptide conjugates”);
(e) mixing the product of step (d) with the target nucleic acid to identify a subgroup of RNA-cDNA-peptide conjugates (hereinafter as “target-specific conjugates”) that are bound to the target nucleic acid;
(f) eluting the cDNAs from the target-specific conjugates of step (e);
(g) subjecting the product of step (f) to PCR to produce a plurality of DNAs (hereinafter as “target-selected DNAs”);
(h) repeating steps (a) to (g) at least 5 times, in which except in the first run of steps (a) to (g), the in vitro transcription in each repeat is performed by using the product of step (g) in a previous run of steps (a) to (g), so as to produce a selected cDNA eluted from the target-specific conjugates of step (f) in the final repeat; and
(i) determining the nucleotide sequence of the selected cDNA of step (h) so as to identify the peptide specific to the target nucleic acid.

According to some exemplary embodiment, each DNA of the DNA library comprises the nucleotide sequence of SEQ ID NO: 14, which, as described above, comprises 12 randomized nucleotide sequences of “MNN”, where M is nucleotide “A” or “C”, and N is any of nucleotide “A”, “T”, “C” or “G”.

In the step (a), the DNA library is subjected to in vitro transcription so as to produce RNAs. According to some embodiments, the in vitro transcription is carried out by mixing DNAs (serving as DNA templates) with RNA polymerase (e.g., T7, SP6 or T3 RNA polymerase), NTPs (including ATP, UTP, CTP and GTP) and reaction buffer (e.g., T7, SP6 or T3 reaction buffer), followed by incubating at an appropriate temperature (e.g., 37° C.) for a period of time (e.g., 2-3 hours). Optionally, the reaction buffer may contain one or more salts (e.g., MgCl₂), reducing agent (e.g., dithiothreitol, DTT), RNase inhibitor or a combination thereof, so as to improve the transcription efficiency. A skilled artisan may adjust the reactants, their concentrations and reaction parameters (e.g., temperature and reaction time) in accordance with intended purposes. The thus-produced RNAs respectively have the nucleotide sequences corresponding to the DNAs of the DNA library.

In the step (b), the RNAs of step (a) is mixed with a puromycin-linker (Pu-linker), a widely used linker in the art, especially useful in mRNA/cDNA display and peptide selection methods. In general, the puromycin-linker comprises in its structure, a binding domain, a puromycin molecule, and a flexible linking domain (e.g., an ethyleneglycol spacer) linking the puromycin molecule to the binding domain. The binding domain comprises a nucleotide sequence complementary to the 3′ terminal region of a target RNA, and accordingly, may hybridize with the 3′ end of the target RNA under a suitable hybridization condition (e.g., incubating at 37° C. for 30-60 minutes). Alternatively, the puromycin-linker may be linked to the 3′ end of a target RNA via a chemical reaction, for example, copper catalysis, ruthenium catalysis, or strain-promoted alkyne-azide cycloaddition. See, for example, Yuki Mochizuki et. al., One-Pot Preparation of mRNA/cDNA Display by a Novel and Versatile Puromycin-Linker DNA, ACS Comb. Sci. (2011), 13: 478-485; Yuki Mochizuki et al., A pull-down method with a biotinylated bait protein prepared by cell-free translation using a puromycin linker, Analytical Biochemistry (2013), 434: 93-95; U.S. Pat. No. 8,445,413 B2; or U.S. Pat. No. 11,345,912 B2. The contents of these publications and application are incorporated herein by reference in their entirety.

In step (c), the product of step (b) (i.e., puromycin-attached RNAs) is subjected to in vitro translation so as to produce RNA/peptide conjugates. As known in the art, the puromycin molecule is a structural analog of aminoacyl-tRNA that can be fused to a nascent polypeptide by peptidyl transferase activity in ribosomes. Thus, after the puromycin molecule is covalently bonded with the 3′ end of the target RNA via the binding domain as described in step (b), the ribosome on the RNA (mRNA) template could stall at the connecting point between the RNA and puromycin-linker during translation reaction, and the puromycin attached to the 3′ end of the puromycin-linker can enter the ribosomal A site and then be fused to the nascent polypeptide chain. In this case, the RNA can be covalently bonded with the corresponding nascent polypeptide chain via the puromycin molecule. See, for example, Yuki Mochizuki et. al., One-Pot Preparation of mRNA/cDNA Display by a Novel and Versatile Puromycin-Linker DNA, ACS Comb. Sci. (2011), 13: 478-485; or Yuki Mochizuki et al., A versatile puromycin-linker using cnvK for high-throughput in vitro selection by cDNA display, Journal of Biotechnology (2015), 212. The contents of these publications and application are incorporated herein by reference in their entirety.

According to certain embodiments of the present disclosure, the in vitro translation is carried out by using a cell-free translation system reconstituted from the purified components necessary for the translation of prokaryotic cells (e.g., E. coli) or eukaryotic cells (e.g., rabbit reticulocytes or HeLa cells). Basically, the cell-free translation system may be prepared as crude extracts containing all the macromolecular components (e.g., 70S or 80S ribosomes, tRNAs, aminoacyl-tRNA synthetases, initiation, elongation and termination factors, etc.) required for translation of exogenous RNA. To ensure efficient translation, each extract may optionally be supplemented with amino acids, energy sources (e.g., ATP, GTP), energy regenerating systems (e.g., creatine phosphate and creatine phosphokinase for eukaryotic systems, and phosphoenol pyruvate and pyruvate kinase for the E. coli lysate), and other co-factors (e.g., Mg₂⁺, K⁺, etc.). The mixture containing the RNA template (e.g., the present RNA/peptide conjugates) and the extracts/supplements is then incubated at an appropriate temperature (e.g., 30° C.) for a period of time (e.g., 90 minutes). A skilled artisan may adjust the reactants, their concentrations and reaction parameters (e.g., temperature and reaction time) in accordance with intended purposes. The thus-translated peptides respectively have the amino acid sequences corresponding to the RNAs of the RNA/peptide conjugates.

In step (d), the RNA/peptide conjugates of step (c) are subjected to reverse transcription so as to produce RNA/cDNA/peptide conjugates. The methods and conditions for reverse transcribing an RNA template into its corresponding cDNA are known by the person having ordinary skill in the art; for the sake of brevity, the detailed descriptions are omitted herein. According to some working examples of the present disclosure, the reverse transcription is carried out by mixing the RNA/cDNA/peptide conjugates with reverse transcriptase, reverse primers, dNTPs (including dATP, dTTP, dCTP and dGTP) and reaction buffer (e.g., Tris-HCl), and then incubating at an appropriate temperature (e.g., 42° C.) for a period of time (e.g., 30 minutes). Depending on desired purposes, the reaction buffer may contain one or more salts (e.g., MgCl₂ and/or LiCl), reducing agent (e.g., DTT), RNase inhibitor or a combination thereof, thereby improving the transcription efficiency. A skilled artisan may adjust the reactants, their concentrations and reaction parameters (e.g., temperature and reaction time) in accordance with intended purposes. The thus-transcribed cDNAs respectively have the nucleotide sequences corresponding to the RNAs of the RNA/peptide conjugates, and accordingly, are capable of hybridizing with the RNAs of the RNA/peptide conjugates, and forming the RNA/cDNA/peptide conjugates. Each RNA/cDNA/peptide conjugate in its structure comprises, a RNA-cDNA duplex (having nucleotide sequences complementary to each other), and a peptide (translated from the RNA) covalently bonded with the RNA via the puromycin molecule.

Optionally, the RNA/cDNA/peptide conjugates of step (d) are subjected to a negative selection via mixing with streptavidin-coated magnetic beads (e.g., streptavidin-coated iron beads) (step (d-1)), and then subjecting to centrifugation or magnetic field (step (d-2)), thereby removing the RNA/cDNA/peptide conjugates with non-specific binding affinity. According to certain embodiments, the mixture of step (d-1) is centrifuged at low speed (e.g., 100, 200, 300, 400 or 500 rpm) for a period of time (e.g., 1-2 hours) to precipitate the magnetic beads and beads-bound RNA/cDNA/peptide conjugates. According to alternative embodiments, the mixture of step (d-1) is exposed to a magnetic field to achieve the same purpose.

The RNA/cDNA/peptide conjugates of step (d) or (d-2) are then subjected to a positive selection via mixing with the target nucleic acid to identity the RNA/cDNA/peptide conjugates exhibiting binding affinity to the target nucleic acid (i.e., the target-specific conjugates; step (e)).

According to some exemplary embodiments of the present disclosure, the target nucleic acid is linked to a biotin molecule (i.e., being a biotinylated target nucleic acid). In this case, the biotinylated target nucleic acid and the RNA/cDNA/peptide conjugates bound thereto (i.e., the target-specific conjugates) may be captured by streptavidin-coated magnetic beads (e.g., streptavidin-coated iron beads; step (e-1)) via the interaction between the peptide and biotinylated target nucleic acid, and the streptavidin-biotin interaction between the biotinylated target nucleic acid and streptavidin-coated magnetic beads.

Depending on desired purpose, the mixture of the RNA/cDNA/peptide conjugates, biotinylated target nucleic acid and streptavidin-coated magnetic beads may be separated via centrifugation or magnetic field (step (e-2)). According to certain embodiments, the mixture is centrifuged at low speed (e.g., 100, 200, 300, 400 or 500 rpm) for a period of time (e.g., 1-2 hours) to precipitate the magnetic beads and beads-bound molecules (i.e., the target nucleic acid and target-specific conjugates). According to alternative embodiments, the mixture is exposed to a magnetic field to achieve the same purpose.

As could be appreciated, the target nucleic acid and the RNA/cDNA/peptide conjugates bound thereto (i.e., the target-specific conjugates) may alternatively be separated by other methods known to pull-down molecules, for example, antibody and antigen interaction.

In the step (f), the cDNA are eluted from the target-specific conjugates with the aid of an elution buffer, which disrupts the binding of molecules, including the biotinylated target nucleic acid, magnetic beads, and the RNAs, cDNAs and peptides of the target-specific conjugates. According to some embodiments of the present disclosure, the elution buffer is an alkaline solution containing a chelating agent, for example, ethylenediaminetetraacetic acid (EDTA).

Next, in the step (g), double-stranded DNAs (dsDNAs) corresponding to the eluted cDNAs of step (f) are produced by PCR. The steps and conditions for producing dsDNAs from cDNAs via PCR are known by the person having ordinary skill in the art; for the sake of brevity, the detailed descriptions are omitted herein. According to certain working examples of the present disclosure, the PCR is carried out by mixing the cDNAs of step (f) (serving as templates) with DNA polymerase, forward and reverse primers, dNTPs (including dATP, dTTP, dCTP and dGTP) and reaction buffer (e.g., Tris-HCl). Depending on intended purposes, the reaction buffer may contain one or more salts (e.g., MgCl₂).

As described in the step (h), the dsDNAs produced by step (g) may serve as the transcription templates of the next round of selection, i.e., the templates for in vitro transcription of step (a). According to some embodiments, steps (a) to (g) are repeated at least 5 times (e.g., 5, 6, 7, 8, 9, 10 or more times) so as to improve the binding specificity of the selected product (i.e., the target-specific peptide and its corresponding cDNA and dsDNA) to the target nucleic acid. According to one exemplary embodiment of the present disclosure, for the purpose of identifying the target-specific peptide, steps (a) to (g) are repeated 6 times. In the embodiment, the target-specific cDNA eluted by step (f) of the last round of selection corresponds to a peptide exhibits binding affinity and/or specificity towards the target nucleic acid.

Accordingly, in the step (i), the nucleotide sequence of the selected cDNA is determined so as to identify the peptide specific to the target nucleic acid. According to certain embodiments, the nucleotide sequence of the selected cDNA is determined by sequencing technology, for example, next generation sequencing (NGS), or Sanger sequencing.

Also disclosed herein are the peptide (i.e., pep11) identified by the present method, and the modified forms of pep11 (i.e., cyclic pep11 and tandem pep11).

According to some embodiments, the pep11 comprises the amino acid sequence of SEQ ID NO: 1, i.e., comprising the amino acid sequence 100% identical to SEQ ID NO: 1.

As its name implies, the cyclic pep11 is the cyclic form of pep11 that has a cyclic ring structure. The ring structure is formed by connecting the amino and carboxyl ends of pep11, i.e., the amino group of the “T” residues at position 1 of SEQ ID NO: 1 and the carboxyl group of the “R” residues at position 12 of SEQ ID NO: 1, via forming an amide bond therebetween.

The tandem pep11 comprises two binding domains linked via a peptide linker, wherein each of the binding domains comprises the amino acid sequence of SEQ ID NO: 1. Depending on desired purpose, the peptide linker may be any linkers useful in linking two peptides, for example, the linker comprising the “G” and “S” residues. According to some exemplary embodiments, the peptide linker comprises the amino acid sequence at least 85% identical to “(GTGSGA)₃” (SEQ ID NO: 2); preferably, at least 90% identical to SEQ ID NO: 2; more preferably, at least 95% identical to SEQ ID NO: 2. According to one specific example, the peptide linker comprises the amino acid sequence of SEQ ID NO: 2. In the embodiment, the tandem pep11 comprises the amino acid sequence of “TKRKHPHRRKYR-(GTGSGA)₃-TKRKHPHRRKYR” (SEQ ID NO: 3).

According to some examples, each of the pep11, cyclic pep11 and tandem pep11 exhibits binding affinity and specificity towards hTERC rG4. According to some examples, the treatment of the pep11, cyclic pep11 or tandem pep11 regulates hTERC gene expression or activity in cells.

Another aspect of the present disclosure pertains to a method of treating a telomerase-related disease in a subject. The method comprises administering to the subject an effective amount of the peptide (i.e., pep11, cyclic pep11, or tandem pep11).

According to some embodiments of the present disclosure, about 0.01 to 1,000 mg/Kg of the present peptide is administered to the subject, such as 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 550, 600, 650, 700, 750, 800, 850, 900, 950, or 1,000 mg/kg. The dose can be administered in a single aliquot, or alternatively in more than one aliquot. The skilled artisan or clinical practitioner may adjust the dosage or regime in accordance with the physical condition of the patient or the severity of the diseases.

Examples of telomerase-related disease treatable with the present method include, but are not limited to, cancer, Hodgkin's disease, dyskeratosis congenital, and aging.

The subject treatable by the present method is a mammal, for example, human, mouse, rat, guinea pig, hamster, monkey, swine, dog, cat, horse, sheep, goat, cow, and rabbit. Preferably, the subject is a human.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLE

### Materials and Methods

### In Vitro Selection (G4-mRNA Display-Seq)

A single-stranded DNA library (ssDNA library) containing a plurality of ssDNAs was synthesized. The double-stranded DNA (dsDNA) library was obtained by template extension and amplification by subjecting the ssDNA library to PCR. Each DNA of the dsDNA library contained a random region having 12 repeats of the nucleotide sequence “MNN”, where M is nucleotide “A” or “C”, and N is any of nucleotide “A”, “T”, “C” or “G”. The generated dsDNA products (SEQ ID NO: 14) were purified and used for in vitro transcription reaction. In vitro transcription reaction was performed with RNA synthesis kit. After incubating at 37° C. for 2.5 hours, DNase was added and incubated at 37° C. for another 15 minutes. The synthesized RNA library was purified by 10% denaturing polyacrylamide gel electrophoresis (PAGE) and RNA column.

In vitro translation was performed by using a release factor-free translation system (PUREXPRESS® Δ RF123 Kit). The reaction was performed on the 150 μL scale with 600 pmol of the mRNA library and 660 pmol puromycin-linker (in the form of “CCCGCCTCCCGCCCCCCGTCC (SEQ ID NO: 15)-(iSp18)₃-CC-Puromycin”, from 5′ end to 3′ end, wherein iSp18 is an 18-atom hexa-ethyleneglycol spacer) for 30 minutes at 37° C. EDTA was added to the solution of the mRNA-displayed peptide library to dissociate ribosomes. The mRNA-displayed peptide library was reverse transcribed by reverse transcriptase for 30 minutes under 42° C.

To prepare for selection, streptavidin magnetic beads were washed and blocked with 0.1 mg/mL yeast tRNA and 1 mg/mL bovine serum albumin (BSA) at 25° C. for 1 hour. After quenching the reverse transcription reaction with EDTA, the solution was neutralized with Tris-HCl (pH 7.5) and then incubated with blocked beads for negative selection. For positive selection, the biotinylated rG4 target immobilized on magnetic beads was mixed with the supernatant from the negative selection at 25° C. for 30 minutes. After the supernatant was removed, the beads were washed with washing buffer (50 mM Tris-HCl pH 7.5, 150 mM KCl, 0.05% TWEEN® 20) for three times. The cDNAs encoding peptides bound to biotinylated rG4-immobilized beads were eluted with the PCR premixture solution. The selected cDNAs on the beads were eluted at 95° C. for 5 minutes and processed to PCR.

After dsDNA column purification, the dsDNA library was used for in vitro transcription for the next selection round. Six rounds of selection were performed in total. Their corresponding conditions including DNA/RNA library input, puromycin linker input, beads input, incubation time, washing time and PCR cycles are all listed in Table 1.

TABLE 1
Conditions used for the in vitro peptide selection process.

| Mining Cycles | 1 | 2 | 3 | 4 | 5 | 6 |
|---|---|---|---|---|---|---|
| Random pool (pmol) | 600^{a} | 10^{b} | 8^{b} | 6^{b} | 6^{b} | 3^{b} |
| Puromycin Linker (pmol) | 660 | 55 | 44 | 33 | 33 | 16.5 |
| RT primer (pmol) | 30 | 50 | 40 | 40 | 40 | 40 |
| Streptavidin-coated | 1,200 | 100 | 80 | 60 | 150 | 150 |
| Magnetic beads (μg) | | | | | | |
| Target (pmol) | 120 | 10 | 8 | 6 | 15 | 15 |
| Negative selection | / | / | 10, 100 | 10, 100 | 30, 200 | 30, 200 |
| (min, μL) | | | | | | |
| Positive selection | 30, 600 | 30, 200 | 30, 200 | 30,200 | 30, 200 | 30, 200 |
| (min, μL) | | | | | | |
| Washing (μL) | 1000 × 3 | 200 × 5 | 200 × 5 | 200 × 5 | 200 × 5 | 200 × 5 |
| Washing (min) | Pipette | Pipette | Pipette | 25 | 25 | 50 |
| | mix | mix | mix | | | |
| PCR cycles | 7 | 12 | 12 | 11 | 12 | 10 |

^{a}RNA random pool.
^{b}DNA random pool. From the second round of selection, the cDNA obtained from the previous round was used as random pool input in the following round.

After six rounds of selection, the ssDNA library obtained from each round was amplified by PCR reaction to add barcode and linker sequences for next-generation sequencing (NGS) purposes.

### Microscale Thermophoresis (MST) Binding Assay

The RNA was subjected to heat treatment at 75° C. for 5 minutes followed by cooling down to room temperature for 10 minutes for renaturation purposes. For binding affinity and selectivity test, reaction mixtures containing 50 nM FAM-labelled RNA, 150 mM KCl, 1 mM MgCl₂, 25 mM Tris-HCl (pH 7.5) and varying concentrations of peptide were prepared and incubated at 37° C. for 30 minutes. The samples were then loaded into MST capillary tubes, and the measurements were taken at 25° C. using blue light mode and the binding affinity mode. The data obtained from these measurements were analyzed using MST nano temper analysis (nta) analysis software. The curve fitting and dissociation constant (Kd) determination were carried out by software using the one site-specific binding model.

### Filter Binding Assay

In the binding affinity and selectivity tests, reaction mixtures containing 5 nM biotin-labeled RNA, 25 mM Tris-HCl (pH 7.5), 150 mM KCl, 1 mM MgCl₂ and varying concentrations of peptides were prepared in 50 μL and incubated at 37° C. for 30 minutes before loaded onto an apparatus containing a nitrocellulose membrane (top) and nylon membrane (bottom). The nylon membrane was rinsed with 0.5× Tris-borate-EDTA (TBE) buffer for 5 minutes, and the nitrocellulose membrane was rinsed with 0.5 M KOH for 15 minutes at 4° C. and then rinsed with 0.5×TBE buffer for one time prior to use. The membranes were washed with binding buffer for three times before and after applying the sample to the apparatus. The crosslinking step was carried out by exposing the membranes to UV irradiation at 254 nm, 120,000 microjoules/cm² for 5 minutes. The results were detected using a chemiluminescent nucleic acid detection module kit, and the scanned by an imaging system.

### NA&M-Enhanced Fluorescence

HTERC rG4 (100 nM) in Tris-HCl buffer (25 mM Tris-HCl, pH 7.5, 150 mM KCl, and 1 mM MgCl₂) was heated at 95° C. for 3 minutes and cooled down at 25° C. for 10 minutes. 10 μL of the peptide and rG4 mixture were incubated for 30 minutes at room temperature. Fluorescence spectroscopy was performed using a microplate reader in 384 well plate. The samples were excited at 394 nm and the emission spectra were detected at 610 nm. The plot was carried out by software.

### FRET Melting Assay

FRET-melting assay was performed with 5′ FAM, 3′ TAMRA labelled hTERC rG4. The oligonucleotides were prepared at 0.2 μM, and the peptides were prepared at 0, 0.2, 0.4, 1 or 2 μM final concentration. The samples were excited at 492 nm and the emission spectra were detected at 516 nm in a buffer containing 10 mM lithium cacodylate and 1 mM KCl. The rG4 was heated at 95° C. for 5 minutes and cooled down in room temperature to refold for 10 minutes before adding the peptides. The signal was monitored by real-time PCR detection system with a procedure of 30 minutes at 25° C., then an increase of 0.5° C. every minute until 95° C. Each experimental condition was tested in three duplicates in a volume of 25 μL for each sample. The data were blanked, averaged and smoothed over 2° C. The plot was carried out by software.

### Cell Imaging

HeLa cells were seeded on a 3.5 cm confocal dish 1 day before transfection. FAM-labelled hTERC rG4 was heated to 75° C. for 5 minutes, annealed at 25° C., and then co-transfected with TAMRA-labelled peptide using LIPOFECTAMINE® 2000. Five hours post-transfection, the cells were stained with 50 nM deep red-fluorescent dye (labeling and tracking acidic organelles in live cells) for 30 minutes and 5 μg/mL Hoechst dye (staining nucleic acid) for 15 minutes. The images were monitored using a laser confocal scanning microscope. Fluorescence statistics of cells were analyzed using software.

### Dual-Luciferase Reporter Gene Assay

Wild-type or mutant hTERC rG4 construct was inserted into the HSV-TK promoter at the SacII restriction enzyme site of firefly/renilla dual luciferase reporter vector, psiCHECK-2. Then, wild-type or mutant reporter plasmid was transfected into Hela cells together with peptides (0, 100, 200 nM) using nucleofector. Each well of the 96-well black-wall optical plate was transfected with 30 ng plasmids. The luciferase reporter gene assays were performed after 48 hours of incubation using the dual-luciferase reporter assay kit. The microplate reader was used to measure luciferase activities with renilla luciferase serving as an internal control. The ratio of firefly to renilla was calculated to analyze the data. Three replicates were performed for each measurement, and the error bar was plotted based on the standard deviation. The P-value was calculated using two-tailed unpaired Student's t-tests to evaluate the significance of the samples.

Total RNA Extraction and Quantitative PCR (qPCR)

Wild-type or mutant reporter plasmids and peptides were co-transfected into Hela cells using the same procedures as for reporter gene assay. After 48 hours of incubation, total RNA was extracted and then subjected to reverse transcription in a 20 μL reaction. Two μL of the cDNA solution from the reverse transcription step was used directly for the qPCR test. All samples were analyzed in triplicate, and the standard deviation was plotted as the error bar. The statistical significance level of results was evaluated using P-values obtained from two-tailed unpaired student's t-tests.

### Example 1 Selection of G4-Binding Peptides by Using the Present G4-mRNA Display-Seq Platform

To develop the mRNA display platform for the G4 target of interest, 12 amino acid random library was designed. As described in “Materials and Methods”, the NNK random library was used, as it was reported to have the benefit of encoding all 20 amino acids over the NNN format. Human telomerase RNA (hTERC) rG4 was used as the target sequence for G4-mRNA display-Seq platform development. This rG4 adopts a parallel topology, and recent studies have shown that this rG4 forms in vitro and in cells. Six rounds of positive selection were conducted with increasing stringency in peptide selection enrichment of peptides specific to the hTERC rG4 target. Additionally, negative selection was introduced, involving the removal of nonspecific binders. To allow more peptide candidates to be identified and analyzed, cDNA library preparation and NGS were conducted, which greatly facilitated the G4-binding peptide discovery.

Based on the NGS results, several enriched peptides were selected for binding tests using microscale thermophoresis (MST). Pep11 (SEQ ID NO: 1), a de novo G4-binding peptide, that exhibited the most promising binding to hTERC rG4 (Kd=1.37±0.22 μM) was selected for further characterization. The binding selectivity tests of pep11 were performed using different rG4s (parallel) and dG4s that can fold into various topologies, including c-MYC dG4 (parallel; SEQ ID NO: 8), c-kit1 dG4 (parallel; SEQ ID NO: 9), HRAS-1 dG4 (antiparallel; SEQ ID NO: 10), and hTelo G4 (mixed parallel/antiparallel; SEQ ID NO: 11), and non-G4 targets such as scramble G-rich sequence, DNA hairpin and RNA hairpin. According to the results, only rG4s exhibited observable binding to pep11, suggesting that the binding is specific to the rG4 targets (data not shown). These results demonstrated that the G4-mRNA display-Seq platform can select novel short peptides for the G4 target of interest.

### Example 2 Characterization of Modified Pep11

### 2.1 Binding Affinity and Specificity

To further improve the G4 binding properties, two modified versions of pep11, tandem and cyclic pep11, were designed. In structure, the tandem pep11 comprised two pep11 sequences (SEQ ID NO: 1) linked by a linker of SEQ ID NO: 2, and the cyclic pep11 consisted of the amino acid sequence of SEQ ID NO: 1 and was present in a cyclic form.

The MST results indicated that the binding affinity to hTERC rG4 was significantly enhanced for both versions, with Kd values of 449±62 and 377±35 nM, respectively (data not shown). To verify the binding results, filter binding assays were further carried out with both hTERC rG4 wild-type (WT; having the nucleotide sequence of “GGGUUGCGGAGGGUGGGCCU”; SEQ ID NO: 6) and hTERC rG4 mutant (MUT; having the nucleotide sequence of “GAAUUGCGGAGAAUGAACCU”; SEQ ID NO: 7). The results confirmed that both tandem and cyclic pep11 bound to hTERC rG4 WT but not MUT (data not shown), suggesting that the modified peptides were specific to G4 structures.

Next, the binding selectivity test was performed using different rG4s, dG4s, and non-G4s. The results indicated that the tandem pep11 bound to other rG4s but not to non-G4s and weakly to dG4s (data not shown). The same analysis was applied on the cyclic pep11, and the results were similar to those of the tandem pep11 (data not shown), suggesting that both versions exhibited great rG4 selectivity.

Overall, the modification and optimization of the rG4-binding peptide construct led to a substantial improvement in rG4 binding affinity, indicating that the rational design of the peptide derived from G4-mRNA display-Seq platform can significantly enhance peptide-rG4 target binding while maintaining selectivity.

### 2.2 Structural Features

The structural features of the rG4-peptide interaction were investigated in the example. Each amino acid residue of pep11 was mutated to be an alanine (A) residue. The data indicated that the mutation of some positively charged amino acids strongly affected the binding ability (data not shown). Further, the results of ligand-enhanced fluorescence assays demonstrated that the fluorescence signals were consistent with or without the peptides, indicating no conformational change of G4 upon peptide binding. RHAU53 (having the amino acid sequence of “SMHPGHLKGREIGMWYAKKQGQKNKEAERQERAVVHMDERREEQIVQLLNSVQA K”; SEQ ID NO: 4), which was reported to interact with rG4s while maintaining their structure, was used as a control that exhibited a similar result (data not shown). Higher melting temperatures were observed by increasing the concentrations of peptides, suggesting the stabilization of hTERC rG4 in the presence of pep11, tandem pep11, or cyclic pep11.

### 2.3 Binding Activity in Live Cells

To assess the ability of the rG4-specific peptides to recognize rG4 structures in live cells, the binding between TAMRA-labeled peptides and hTERC rG4 in vitro was examined, and the peptide (i.e., tandem pep11 or cyclic pep11) was co-transfected with hTERC rG4 WT or MUT into cells. The results indicated clear colocalization of TAMRA signals with the fluorescent foci in the cells transfected with hTERC rG4 WT, demonstrating the interaction of the peptide (i.e., tandem pep11 or cyclic pep11) with rG4 structures in live cells (data not shown). In contrast, few foci were observed in the cells transfected with hTERC rG4 MUT (data not shown), indicating that the interaction was rG4-specific.

Altogether, these results support the ability of the tandem pep11 and cyclic pep11 to recognize and interact with rG4 structures in live cells and provide further evidence for the specificity and effectiveness of the rG4-targeting peptides.

2.4 Effect of Modified Pep11 on Modulating rG4-Medicated Gene Expression

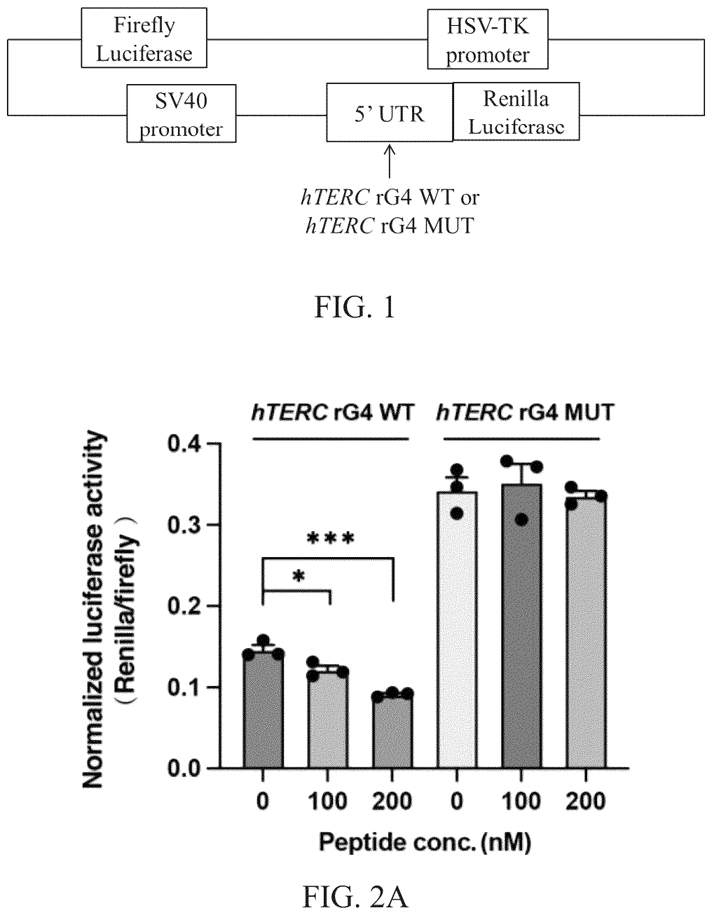

After confirming that the rG4-targeting peptides can interact with rG4 structures in live cells, their effect on modulating rG4-mediated gene expression was examined in the example. Dual luciferase reporter gene assay was conducted in HeLa cells, in which hTERC rG4 WT motif (SEQ ID NO: 12) or hTERC rG4 MUT motif (SEQ ID NO: 13) was inserted in the 5′ untranslated region (5′ UTR) of the renilla luciferase gene region separately, and the firefly luciferase gene was used as a normalized gene (FIG. 1).

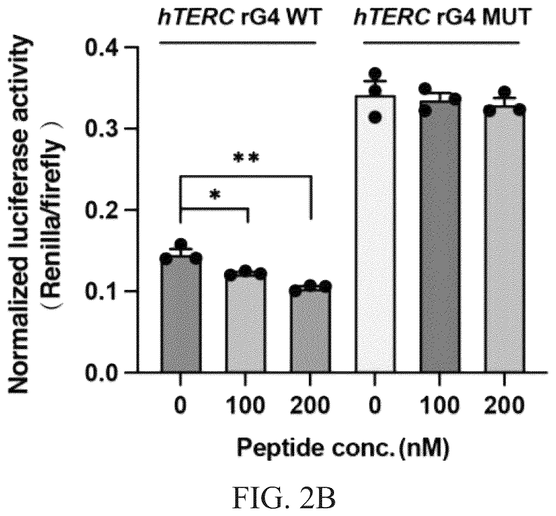

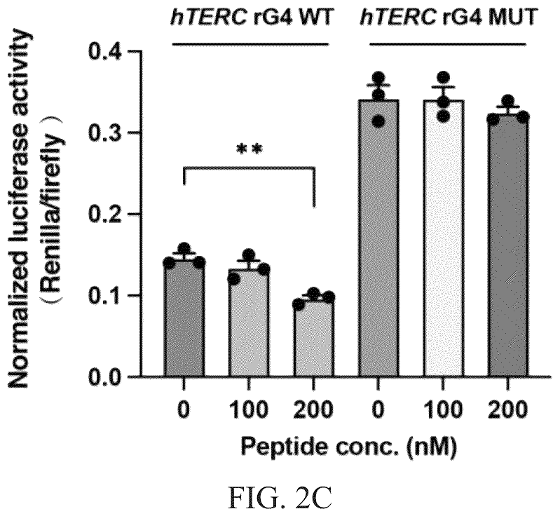

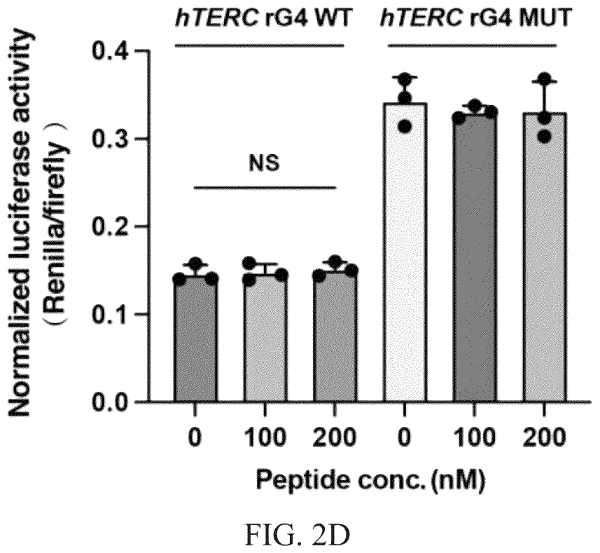

Without rG4-targeting peptides, the hTERC rG4 WT construct exhibiting 2.34±0.16-fold lower normalized luciferase activity than the hTERC rG4 MUT construct, suggesting that the hTERC rG4 motif functions in cells and negatively regulates the gene expression (FIGS. 2A-2D). Importantly, for the WT construct but not the MUT construct, the inhibition extended with increasing concentration of tandem pep11 or cyclic pep11 addition (FIGS. 2A and 2B), highlighting that both peptides regulate the gene activity in cells. Moreover, RHAU53 was used as a positive control peptide, and the data indicated that the effects of both tandem and cyclic pep11 were comparable to that of RHAU53 (FIGS. 2A-2C). The negative control, pep71 (having the amino acid sequence of “AHPLTKPPKYHH”; SEQ ID NO: 5), did not impact the hTERC rG4 WT or MUT construct (FIG. 2D), indicating that the targeting was rG4-specific. In addition, reverse transcription quantitative PCR (RT-qPCR) was also conducted, and no significant changes were observed at the mRNA level (data not shown), demonstrating that the inhibition effect was at the translational level but not at the transcriptional level. These findings provide substantial evidence that the tandem and cyclic pep11 were capable of suppressing translation by targeting the rG4 of interest.

It will be understood that the above description of embodiments is given by way of example only and that various modifications may be made by those with ordinary skill in the art. The above specification provides a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method of identifying a peptide specific to a target nucleic acid from a deoxyribonucleic acid (DNA) library, wherein the DNA library comprises a first plurality of DNAs respectively having randomized sequences, and the method comprises,
(a) producing a plurality of ribonucleic acids (RNAs) respectively corresponding to the first plurality of DNAs via in vitro transcription;
(b) attaching a puromycin molecule to the 3′ end of each of the plurality of RNAs of step (a) via a linker;
(c) subjecting the product of step (b) to in vitro translation to produce a plurality of RNA-peptide conjugates;
(d) subjecting the product of step (c) to reverse transcription to produce a first plurality of RNA-complementary DNA (cDNA)-peptide conjugates;
(e) mixing the product of step (d) with the target nucleic acid to identify a second plurality of RNA-cDNA-peptide conjugates bound to the target nucleic acid;
(f) eluting the cDNAs from the second plurality of RNA-cDNA-peptide conjugates of step (e);
(g) subjecting the product of step (f) to polymerase chain reaction (PCR) to produce a second plurality of DNAs;
(h) repeating steps (a) to (g) at least 5 times, in which except in the first run of steps (a) to (g), the in vitro transcription in each repeat is performed by using the product of step (g) in a previous run of steps (a) to (g), so as to produce a selected cDNA eluted from the second plurality of RNA-cDNA-peptide conjugates of step (f) in the final repeat; and
(i) determining the nucleotide sequence of the selected cDNA of step (h) so as to identify the peptide specific to the target nucleic acid.

2. The method of claim 1, wherein the target nucleic acid has a G-quadruplex structure.

3. The method of claim 1, wherein the target nucleic acid of step (e) is linked to a biotin molecule, and the method further comprises,
(e-1) capturing the product of step (e) via a streptavidin-coated bead prior to step (f).

4. The method of claim 3, further comprising,
(e-2) subjecting the product of step (e-1) to centrifugation or magnetic field prior to step (f).

5. The method of claim 3, further comprising,
(d-1) mixing the product of step (d) with the streptavidin-coated bead prior to step (e).

6. The method of claim 5, further comprising,
(d-2) subjecting the product of step (d-1) to centrifugation or magnetic field prior to step (e).

7. A peptide identified by the method of claim 1 comprising the amino acid sequence of SEQ ID NO: 1.

8. The peptide of claim 7, wherein the peptide is in cyclic form.

9. The peptide of claim 7, wherein the peptide is in tandem form and comprises two binding domains linked via a peptide linker, wherein each of the two binding domains comprises the amino acid sequence of SEQ ID NO: 1.

10. The peptide of claim 9, wherein the peptide linker comprises the amino acid sequence of SEQ ID NO: 2.

11. The peptide of claim 10, wherein the peptide comprises the amino acid sequence of SEQ ID NO: 3.

12. A method of treating a telomerase-related disease in a subject comprising administering to the subject an effective amount of the peptide of claim 7.

13. The method of claim 12, wherein the telomerase-related disease is cancer, Hodgkin's disease, dyskeratosis congenital, or aging.

14. The method of claim 12, wherein the subject is a human.
